(19) United States
(12) Patent Application Publication
(10) Pub. No.: US 2024/0215907 A1
(43) Pub. Date: Jul. 4, 2024

(54) WEARABLE SPINAL MEASUREMENT DEVICE

(71) Applicants: NATIONAL TSING HUA UNIVERSITY, Hsinchu (TW); NATIONAL TAIWAN UNIVERSITY HOSPITAL HSIN-CHU BRANCH, Zhubei City (TW)

(72) Inventors: TING-JEN YEH, Hsinchu (TW); MING-CHANG HSU, Hsinchu (TW); HSUAN-YU CHEN, Zhubei City (TW)

(21) Appl. No.: 18/537,845

(22) Filed: Dec. 13, 2023

(30) Foreign Application Priority Data
Dec. 30, 2022 (TW) 111150928

(51) Int. Cl. A61B 5/00

(52) U.S. Cl. CPC A61B 5/4561; A61B 5/6823

(57) ABSTRACT

In this invention, a novel wearable device is proposed for long-time measurement of user's spine posture and alignment. Mechanically, the device primarily comprises an actuation module, a measurement mechanism, and a processing module positioned on the wearing component. The measurement mechanism includes a primary measurement unit and at least one secondary measurement unit. These measurement units are interconnected in series, forming an underactuated mechanism, and are actuated by the actuation module. On each first primary/secondary rotational axis, at least one angular sensor is employed to measure the angle change relative to the adjacent measurement unit. The processing module is electrically connected to both the actuating module and at least one angular sensor, allowing it to receive angle change signals from the sensors and transmit a measurement signal from the wearable spinal measurement device.

### CROSS-REFERENCE TO RELATED APPLICATION

This non-provisional application claims priority under 35 U.S.C. § 119(a) on Patent Application No(s). 111150928 filed in Taiwan, R.O.C. on Dec. 30, 2022, the entire contents of which are hereby incorporated by reference.

### BACKGROUND OF THE INVENTION

### 1. Field of the Invention

The present disclosure relates to a measurement device, and more particularly, to a wearable spinal measurement device.

### 2. Description of the Related Art

The 2022 World Population Prospects report projects an increase in the proportion of individuals aged over 65 years from 9.7% to 11.7% between 2022 and 2030. This demographic shift towards an aging population has raised concerns about the quality of life among the elderly, with a particular focus on issues related to falls and subsequent fractures. These incidents can lead to a gradual loss of independence and an increase in the bedridden rate. Clinical studies have indicated that the imbalance of spinal alignment and postural instability are crucial early indicators of falls in the elderly. Therefore, monitoring spinal posture and alignment during daily activities provides valuable information for clinical diagnosis in balancing analysis and degeneration prediction.

Presently, plain radiography is the standard method for examining the posture and alignment of the spine in the clinical pathway, as it provides X-ray images with accurate geometric and structural information. However, to mitigate the risk of injury from prolonged radiation exposure, plain radiography can only offer a few instantaneous images, making it unsuitable for capturing continuous spinal motion crucial for diagnosing preliminary spinal symptoms.

As an alternative method, Roveda, L. et al. utilized a motion capture system to record the motion of reflective markers attached to the back of the patient, constructing continuous spinal posture and alignment. (Roveda, L., Savani, L., Arlati, S., Dinon, T., Legnani, G., and Tosatti, L. M., 2020, “Design methodology of an active back-support exoskeleton with adaptable backbone-based kinematics,” International Journal of Industrial Ergonomics, 79, p. 102991).

Plamondon, A. et al. employed two inertial sensors to measure the three-dimensional posture and alignment of the trunk, compensating for measurement errors due to magnetometers by adding a potentiometer to measure the relative rotation between two sensors (Plamondon, A., Delisle, A., Larue, C., Brouillette, D., McFadden, D., Desjardins, P., and Lariviere, C., 2007, “Evaluation of a hybrid system for three-dimensional measurement of trunk posture in motion,” Applied Ergonomics, 38(6), pp. 697-712).

In the research conducted by Sardini, E. et al., an impedance sensor is sewn directly onto the T-shirt. The impedance change of the sensor provides rough posture and alignment data. Fodor, C. et al. applied a piezo-resistive transducer to sense its contact force with the trunk to distinguish spinal posture and alignment (Sardini, E., Serpelloni, M., and Pasqui, V., 2015, “Daylong sitting posture measurement with a new wearable system for at-home body movement monitoring,” In 2015 IEEE International Instrumentation and Measurement Technology Conference (I2MTC) Proceedings, IEEE, pp. 652-657; Fodor, Cristian, et al. “Device for telemonitoring of spine flexion during cervical kyphosis treatment.” 2019 11th International Symposium on Advanced Topics in Electrical Engineering (ATEE). IEEE, 2019).

In an attempt to construct the entire profile of the spine using only a few sensors, Voinea, G.-D. et al. and Wong, W. Y. et al. combined orientation angles measured from several inertial sensors with a mathematical model to calculate the curvature of the spine. (Voinea, G.-D., Butnariu, S., and Mogan, G., 2016, “Measurement and geometric modeling of human spine posture for medical rehabilitation purposes using a wearable monitoring system based on inertial sensors,” Sensors, 17(1), p. 3; Wong, W. Y., and Wong, M. S., 2008, “Trunk posture monitoring with inertial sensors,” European Spine Journal, 17, pp. 743-753).

### BRIEF SUMMARY OF THE INVENTION

The aspects and advantages of the embodiments in this disclosure will be explained partly in the following description. One can gain insights from this description or acquire knowledge through practical application of the embodiments.

The human spine is composed of 17 vertebral segments, each possessing three rotational degrees of freedom. As a consequence, the development of wearable devices for spinal applications presents more challenges compared to those for limb applications. This is primarily due to the spine's high degrees of freedom (DOFs) and the restricted inter-joint spaces. In the current research on wearable spinal measurement devices, there is a tendency to overlook the high DOF characteristics of the spine. Instead, simplistic approaches are favored which employ a few miniaturized electronic sensors to capture spinal motion at specific locations. Within the realm of existing wearable spinal measurement devices, certain designs utilize impedance sensors or piezo-resistive transducers for estimating spinal posture. While structurally simple, these designs fall short in delivering a comprehensive measurement of spinal posture. On the other hand, a design that incorporates two inertial sensors and a potentiometer achieves a three-dimensional correlation between the upper and lower trunk but fails to capture the complete posture of user's spine. The utilization of accelerometers in combination with mathematical estimation and modeling, though a prevalent approach, introduces inherent errors associated with the estimation-based model. Furthermore, accelerometers are susceptible to influences from dynamic motion, making them primarily suitable for static posture measurements. In the realm of non-wearable imaging technologies, X-ray applications, while suitable for detailing spinal posture, fall short in providing continuous, real-time tracking capabilities over extended durations. 3D motion capture systems, despite their proficiency in dynamic motion and posture detection, suffer from cost constraints and implementation challenges in diverse settings.

Due to these existing challenges in acquiring continuous spinal posture, this disclosure introduces a novel wearable spinal measurement device. This innovative device not only attains adaptability to the high degrees of freedom inherent in the spine but also ensures constant adherence to the user's spinal contours for accurate posture measurement. Furthermore, it provides uninterrupted information on spinal posture over prolonged durations, all within a lightweight structure and at a cost conducive to widespread applicability in diverse settings.

Accordingly, the present disclosure provides a wearable spinal measurement device, comprising: a wearing component; an actuation module disposed on the wearing component, wherein the actuation module includes a transmission element; a measurement mechanism disposed on the wearing component, the measurement mechanism includes a primary measurement unit including a first primary linkage connected to the transmission element of the actuation module at one end, a second primary linkage pivotally connected at one end to the first primary linkage via a second primary rotational axis, a third primary linkage pivotally connected at one end to the second primary linkage via a third primary rotational axis, a fourth primary linkage pivotally connected at one end to the third primary linkage via a fourth primary rotational axis, and at the other end pivotally connected to the first primary linkage via a first primary rotational axis; at least one angular sensor disposed on the first primary rotational axis for measuring the angle relationship between the first primary linkage and the fourth primary linkage; and a processing module electrically connected to the at least one angular sensor, the processing module receiving angle change signals from the at least one angular sensor and transmitting a measurement signal between the wearable spinal measurement device and the external environment.

In the wearable spinal measurement device described above, the measurement mechanism further includes a plurality of secondary measurement units, each of which comprises a first secondary linkage, a second secondary linkage pivotally connected at one end to the first secondary linkage via a second secondary rotational axis, a third secondary linkage pivotally connected at one end to the second secondary linkage via a third secondary rotational axis, and a fourth secondary linkage pivotally connected at one end to the third secondary linkage via a fourth secondary rotational axis, and pivotally connected at the other end to the first secondary linkage via a first secondary rotational axis, wherein the first secondary linkage of one of the plurality of secondary measurement units is coaxially disposed with the third primary linkage of the primary measurement unit, and the first secondary linkage of the other secondary measurement units is coaxially disposed with the third secondary linkage of the adjacent secondary measurement units, wherein the plurality of secondary measurement units are arranged along a line connecting the first primary rotational axis to the fourth primary rotational axis of the primary measurement unit.

In an embodiment of the wearable spinal measurement device, the actuation module is a torsion spring.

In an embodiment of the wearable spinal measurement device, the number of secondary measurement units is matched to the number of vertebral segments (hereinafter, also “spinal bones”) of the user's spine.

In an embodiment of the wearable spinal measurement device, the number of secondary measurement units is matched to the number of vertebral segments whose postures are to be measured.

In an embodiment of the wearable spinal measurement device, the processing module is further electrically connected to the actuation module, and the processing module outputs a control signal to the actuation module; and the actuation module is a servo motor.

In an embodiment of the wearable spinal measurement device, it further includes a plurality of pressure sensors disposed on the fourth primary rotational axis of the primary measurement unit relative to the third primary linkage at one side and disposed on the fourth secondary rotational axis of each secondary measurement unit relative to the third secondary linkage at one side for measuring contact pressure. The plurality of pressure sensors are electrically connected to the processing module, and the processing module receives contact pressure signals from the plurality pressure sensors and outputs control signals to the actuation module, transmitting measurement signals between the wearable spinal measurement device and the external environment.

In an embodiment of the wearable spinal measurement device, the processing module further includes a processor that adjusts the signals output to the actuation module in real-time based on angle change signals from angular sensors and contact pressure signals from pressure sensors, thereby adjusting the torque of the transmission element, and transmitting measurement signals to the external environment in real-time.

In an embodiment of the wearable spinal measurement device, the measurement mechanism is an underactuated mechanism.

In an embodiment of the wearable spinal measurement device, the angular sensor is a magnetic encoder.

In an embodiment of the wearable spinal measurement device, the angular sensing accuracy of the angular sensor is less than or equal to 0.1 degrees.

In an embodiment of the wearable spinal measurement device, the angular sensor is wirelessly connected to the processing module.

In an embodiment of the wearable spinal measurement device, an inertial sensor is disposed on the actuation module for measuring the coordinates of the actuation module relative to the earth.

In an embodiment of the wearable spinal measurement device, the pressure sensor is a flexible force-sensitive resistor (FSR) pressure sensor.

In an embodiment of the wearable spinal measurement device, the pressure sensor is wirelessly connected to the processing module.

In an embodiment of the wearable spinal measurement device, the processor of the processing module calculates the corresponding spinal posture based on the size information and physical properties of the measurement mechanism and angle information from the angular sensors, and transmits the spinal posture to the external environment.

In an embodiment of the wearable spinal measurement device, the size characteristics and physical properties of the measurement mechanism are calculated by a genetic algorithm based on a mathematical computational model.

In an embodiment of the wearable spinal measurement device, the mathematical computation model includes a static model of a continuous underactuated mechanism, a static model of a continuous underactuated mechanism with gravity, or a static model of a continuous underactuated mechanism with integrated elastic elements.

The wearable spinal measurement device provided by the present disclosure can conform to the user's spinal segments, transmit spinal posture-related information to the external environment, achieve continuous and accurate tracking and measurement of spinal posture over an extended period, and reduce the weight of the spinal measurement device.

These and other features, aspects, and advantages of various embodiments will become better understood with reference to the following description and appended claims. The accompanying drawings, which are incorporated in and constitute a part of this specification, illustrate embodiments of the present disclosure, and together with the description, serve to explain the related principles.

### BRIEF DESCRIPTION OF THE DRAWINGS

FIG. 1A is a schematic block diagram of a wearable spinal measurement device in an embodiment of the present disclosure.

FIG. 1B is a schematic block diagram of a wearable spinal measurement device in an embodiment of the present disclosure.

FIG. 2 is a schematic illustration of a wearable spinal measurement device in an embodiment of the present disclosure.

FIG. 3A is a schematic diagram illustrating the structure of a wearable spinal measurement device in an embodiment of the present disclosure.

FIG. 3B is a schematic illustration of the operation of a wearable spinal measurement device in an embodiment of the present disclosure.

FIG. 4A is a schematic diagram illustrating the structure of a wearable spinal measurement device in an embodiment of the present disclosure.

FIG. 4B is a schematic illustration of the operation of a wearable spinal measurement device in an embodiment of the present disclosure.

FIG. 5 is a schematic block diagram of an embodiment of the wearable spinal measurement device of the present disclosure.

FIG. 6 is a schematic illustration of the operation of an embodiment of the wearable spinal measurement device of the present disclosure.

### DETAILED DESCRIPTION OF THE INVENTION

To facilitate understanding of the objectives, characteristics, and effects of the present disclosure, embodiments together with the attached drawings for the detailed description of the present disclosure are provided.

In order to provide a detailed explanation of the technical content and structural features of the present disclosure, the following description is provided in conjunction with embodiments and accompanying drawings.

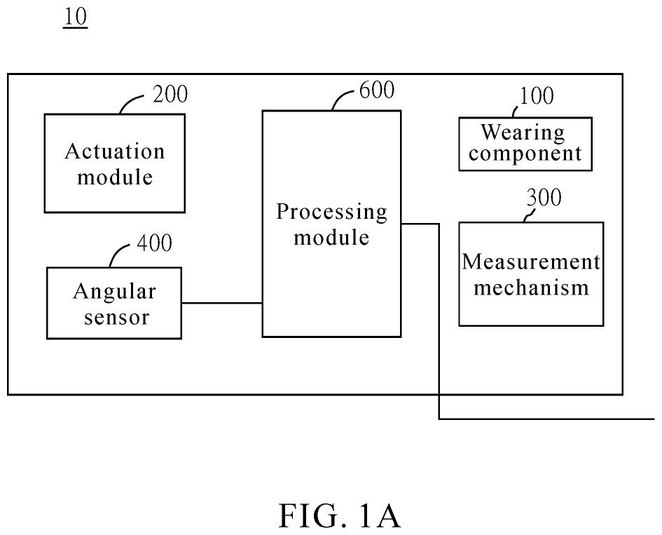

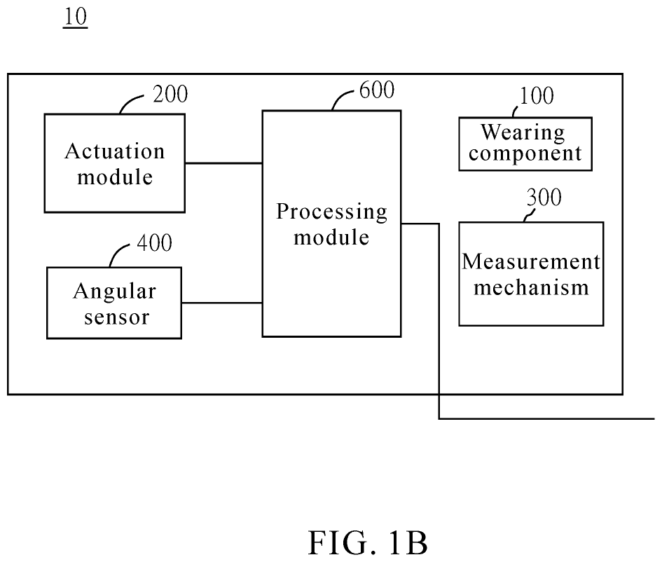

FIG. 1A is a schematic block diagram of a wearable spinal measurement device of an embodiment of the present disclosure. The wearable spinal measurement device 10 includes a wearing component 100, an actuation module 200, a measurement mechanism 300, an angular sensor 400, and a processing module 600 electrically connected to the angular sensor 400. The processing module 600 receives angle change signals from the angular sensor 400 and transmits measurement signals between the wearable spinal measurement device 10 and the external environment. The measurement signals include angle change signals. In some embodiments of the present disclosure, the actuation module 200 is a torsion spring.

FIG. 1B is a schematic block diagram of another embodiment of the wearable spinal measurement device of the present disclosure. The wearable spinal measurement device 10 includes a wearing component 100, an actuation module 200, a measurement mechanism 300, an angular sensor 400, and a processing module 600 electrically connected to both the actuation module 200 and the angular sensor 400. The processing module 600 receives angle change signals from the angular sensor 400, outputs control signals to the actuation module 200, and transmits measurement signals between the wearable spinal measurement device 10 and the external environment. The measurement signals include angle change signals and control signals. In some embodiments of the present disclosure, the actuation module 200 is a servo motor.

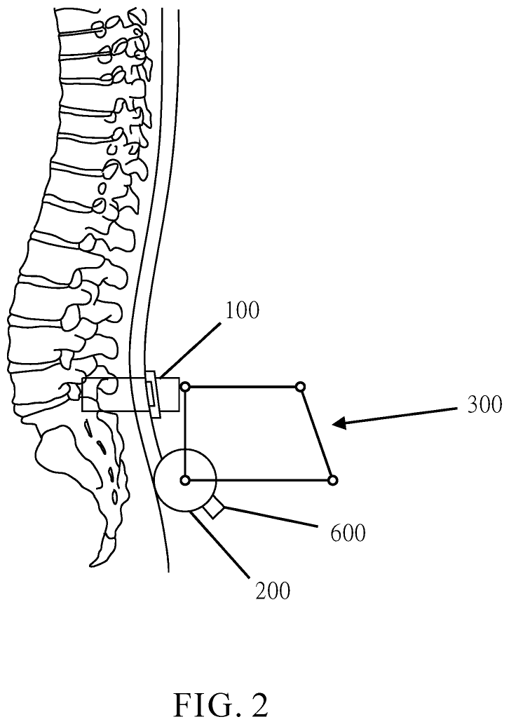

FIG. 2 is a schematic illustration of a wearable spinal measurement device in some embodiments of the present disclosure. The wearable spinal measurement device 10 has a wearing component 100 for wearing on the user's back, an actuation module 200 is disposed on the wearing component 100, and in some embodiments, the actuation module 200 is placed at the bottom of the wearing component 100. The measurement mechanism 300 is disposed on the outer side of the wearing component 100 relative to the user, and the processing module 600 is disposed outside the actuation module 200. It should be noted that the positioning of the processing module 600 is not limited to outside of the actuation module 200; the processing module 600 can also be placed within the wearing component 100 or the measurement mechanism 300. The processing module 600 can be connected via wired or wireless communication and can be placed at any location within the wearable spinal measurement device 10, including being independently positioned outside of the device.

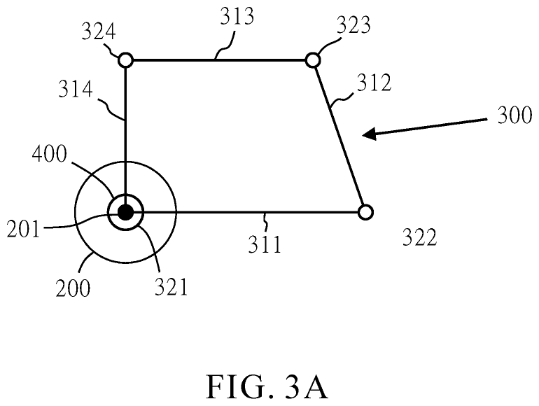

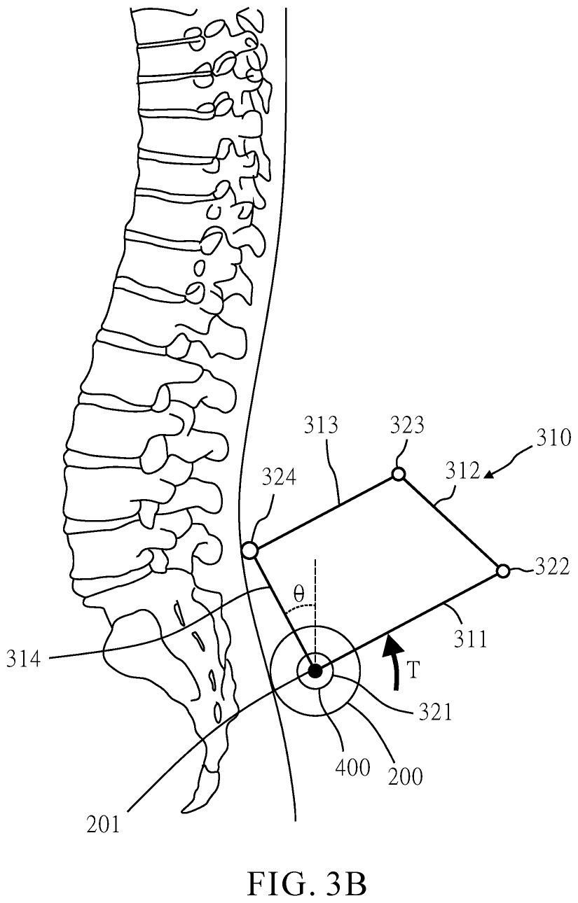

FIG. 3A is a schematic diagram illustrating the structure of a wearable spinal measurement device in some embodiments of the present disclosure, further explaining the structural relationship between the actuation module 200 and the measurement mechanism 300. The actuation module 200 includes a transmission element 201, and the measurement mechanism 300 includes a primary measurement unit 310. The primary measurement unit 310 has a first primary linkage 311, one end of which is connected to the transmission element 201 of the actuation module 200. The primary measurement unit 310 also has a second primary linkage 312, one end of which is pivotally connected to the other end of the first primary linkage 311 via a second primary rotational axis 322. The primary measurement unit 310 further has a third primary linkage 313, one end of which is pivotally connected to the other end of the second primary linkage 312 via a third primary rotational axis 323. The primary measurement unit 310 also has a fourth primary linkage 314, one end of which is pivotally connected to the other end of the third primary linkage 313 via a fourth primary rotational axis 324, and the other end is pivotally connected to the first primary linkage 311 via a first primary rotational axis 321. Importantly, the transmission element 201 of the actuation module 200 is only connected to the first primary linkage 311 and exerts torque only on the first primary linkage 311, without applying torque to the fourth primary linkage 314. The first primary rotational axis 321 is coaxially connected to the transmission element 201, but the fourth primary linkage 314 is not directly subjected to forces from the transmission element 201.

As shown in FIG. 3A, in some embodiments of the present disclosure, the wearable spinal measurement device includes angular sensors 400 disposed on the first primary rotational axis 321 to measure angle relationship information between the first primary linkage 311 and the fourth primary linkage 314, and angle change signals of angle relationship information are transmitted to the processing module 600 through electrical connections. In some embodiments of the present disclosure, the actuation module 200 is a servo motor, and the processing module 600 controls the actuation module 200 based on the angle change information measured by the angular sensor 400 on the first primary linkage 311 and the fourth primary linkage 314. The processing module 600 further integrates angle change signals, control signals for the actuation module 200, and other relevant angle change information, and transmits this integrated measurement signal between the wearable spinal measurement device and the external environment. In an embodiment of the present disclosure, the actuation module 200 is a torsion spring, providing the advantage of lightweight and the ability to output a fixed amount of torque without requiring electrical power.

FIG. 3B is a schematic illustration of the operation of a wearable spinal measurement device in some embodiments of the present disclosure. By actuating the actuation module 200 to rotate the transmission element 201 and apply torque to the first primary linkage 311, the first primary linkage 311 is lifted upwards (e.g., denoted as T representing the direction of torque). This action also causes the positions of the second primary linkage 312, the third primary linkage 313, and the fourth primary linkage 314 to change. The fourth primary rotational axis 324 comes into contact with the user's back. By measuring the angle change information (e.g., 0 representing the angle change information of the fourth primary linkage 314 relative to the vertical direction) using angular sensors 400 disposed on the first primary rotational axis 321 and by considering the size characteristics of the measurement mechanism 300, such as the length of the fourth primary linkage 314, the processing module 600 integrates angle information and transmits it to the external environment. This allows for determining the spatial state of the measurement mechanism 300 in three dimensions, thereby achieving the determination of the user's spinal posture when using the wearable spinal measurement device 10. In some embodiments, the processing module 600 can transmit measurement signals to a computer, mobile phone, wearable electronic device, mobile device, medical facility-related system, cloud server, etc., for reference and use by users or healthcare professionals based on the information in the measurement signals.

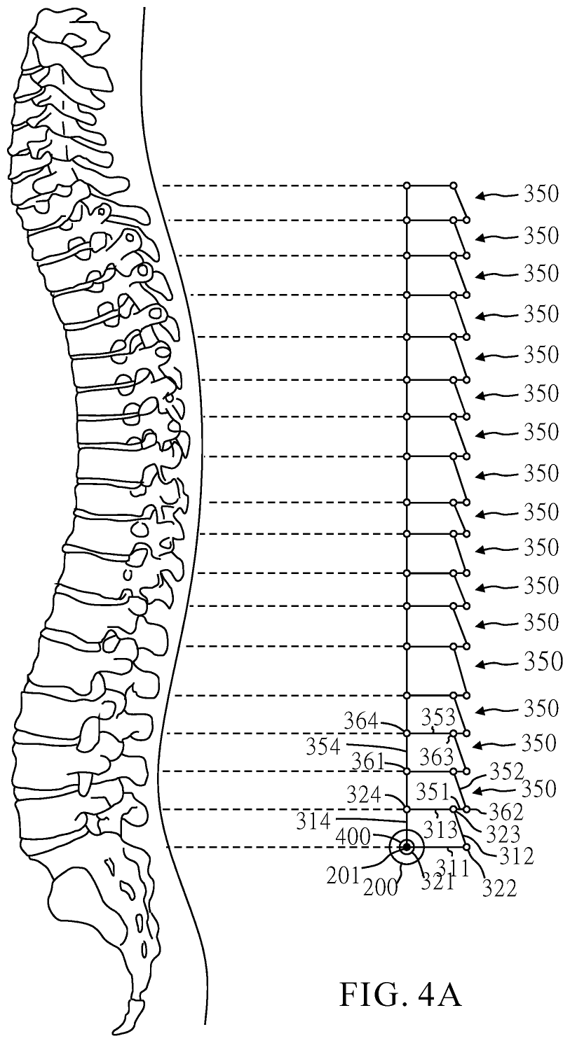

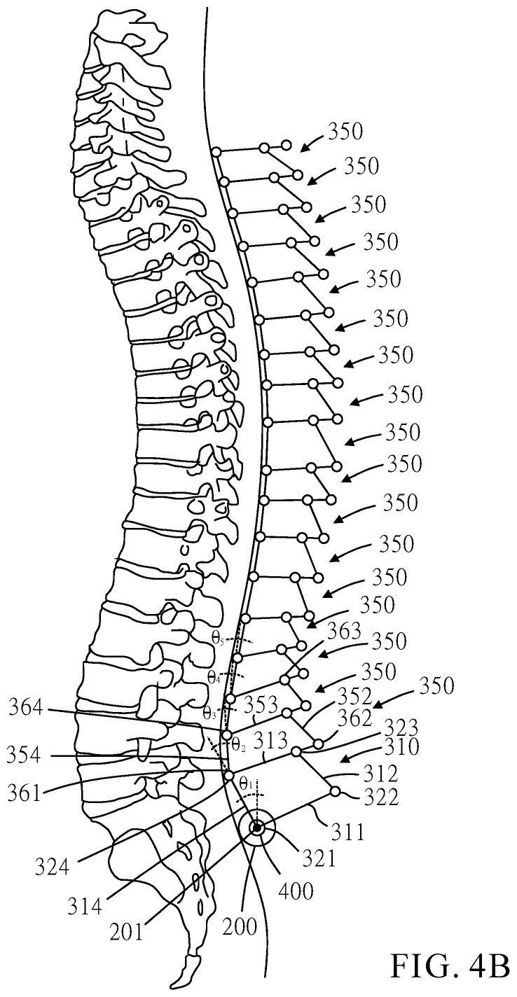

FIG. 4A is a schematic diagram illustrating the structure of a wearable spinal measurement device in some embodiments of the present disclosure. The human spine generally consists of 17 segments. To accommodate various needs of different users, the wearable spinal measurement device of the present disclosure can correspondingly set different numbers of secondary measurement units 350 on top of the primary measurement unit 310. As shown in FIG. 4A, the actuation module 200 includes a transmission element 201, and the measurement mechanism 300 includes a primary measurement unit 310 and plurality secondary measurement units 350. In some embodiments, the measurement mechanism 300 includes one primary measurement unit 310 and sixteen secondary measurement units 350 to correspond to the 17 segments of the human spine. Each secondary measurement unit 350 has a first secondary linkage 351 and a second secondary linkage 352, one end of which is pivotally connected to one end of the first secondary linkage 351 via a second secondary rotational axis 362. Each secondary measurement unit 350 also has a third secondary linkage 353, one end of which is pivotally connected to the other end of the second secondary linkage 352 via a third secondary rotational axis 363. Each secondary measurement unit 350 further has a fourth secondary linkage 354, one end of which is pivotally connected to the other end of the third secondary linkage 353 via a fourth secondary rotational axis 364, and the other end is pivotally connected to the first secondary linkage 351 via a first secondary rotational axis 361. Importantly, the first secondary linkage 351 of one of the plurality secondary measurement units 350 is coaxially arranged with the third primary linkage 313 of the primary measurement unit 310 (i.e., the first secondary linkage 351 and the third primary linkage 313 always maintain a coaxial state). The other first secondary linkages 351 of the remaining secondary measurement units 350 and the third secondary linkages 353 of the adjacent secondary measurement units 350 are also coaxially arranged. It should be noted that the coaxially arranged connecting members can indicate that they share a portion of the connecting members and extend in the axial direction (e.g., the first secondary linkage 351 has a longer length than the third primary linkage 313 and shares the entire third primary linkage 313 and extends further in the axial direction). The plurality of secondary measurement units 350 are arranged in the direction of the connecting lines from the first primary rotational axis 321 to the fourth primary rotational axis 324. That is, above the primary measurement unit 310, the required number of secondary measurement units 350 are sequentially placed to correspondingly measure and track each segment of the spine as needed, achieving the effect of measuring the entire spine.

FIG. 4B is a schematic illustration of the operation of a wearable spinal measurement device in some embodiments of the present disclosure. In some embodiments where the measurement mechanism 300 includes one primary measurement unit 310 and sixteen secondary measurement units 350, when the actuation module 200 positioned at the bottom of the wearable spinal measurement device 10 is actuated, the first primary linkage 311 of the primary measurement unit 310 is subjected to force and lifts upward, causing the second primary linkage 312, the third primary linkage 313, and the fourth primary linkage 314 of the primary measurement unit 310 to move. Due to the coaxial arrangement of the first secondary linkage 351 of the upper adjacent secondary measurement unit 350 with the third primary linkage 313 of the primary measurement unit 310, the first secondary linkage 351, the second secondary linkage 352, the third secondary linkage 353, and the fourth secondary linkage 354 of each adjacent secondary measurement unit 350 are subjected to force and move. The fourth secondary rotational axis 364 of each secondary measurement unit 350 also comes into contact with the user's back. When the fourth primary rotational axis 324 of the primary measurement unit 310 and the fourth secondary rotational axis 364 of each secondary measurement unit 350 are in contact with the user's back, the angular sensors 400 disposed on the first primary rotational axis 321 and the angular sensors 400 disposed on the first secondary rotational axis 361 can obtain angle change information of the first primary rotational axis and each corresponding first secondary rotational axis (e.g., θ1, θ2, θ3, . . . θ16, θ17 represent the angle relationships between adjacent connecting members), and transmit angle change signals from each angular sensor 400 to the processing module 600. Through the processing module 600, the angle change signals obtained by each angular sensor 400 are transmitted to the external environment, and by considering the size characteristics of the measurement mechanism 300 (i.e., the length of the fourth primary linkage 314 of the primary measurement unit 310 and the fourth secondary linkage 354 of each secondary measurement unit 350), the spatial state of the measurement mechanism 300 in three dimensions can be determined. This achieves the measurement of the user's entire 17-segment spine, allowing for real-time and accurate measurement of the user's spinal posture with a convenient and balanced device.

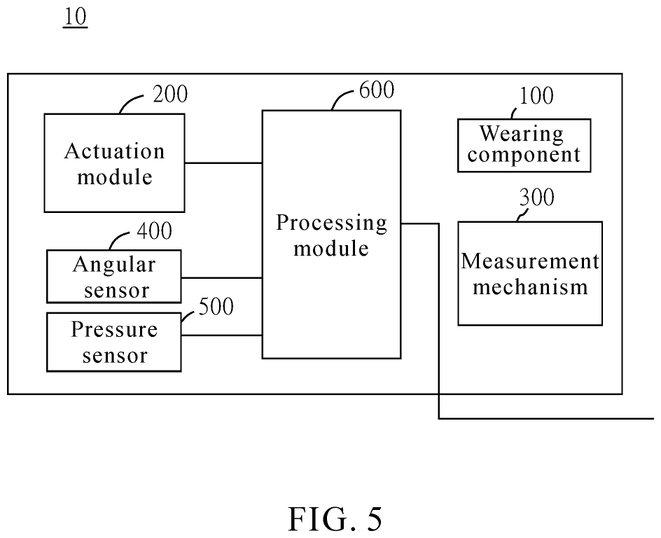

FIG. 5 is a block diagram illustrating another embodiment of the wearable spinal measurement device in the present disclosure. The wearable spinal measurement device 10 comprises wearing components 100, an actuation module 200, a measurement mechanism 300, an angular sensor 400, a pressure sensor 500, and a processing module 600 electrically connected to the actuation module 200, angular sensor 400, and pressure sensor 500. The processing module 600 receives angle change signals from the angular sensor 400 and contact pressure signals from the pressure sensor 500, outputs control signals to the actuation module 200, and transmits integrated angle change signals and contact pressure signals, as measurement signals, between the wearable spinal measurement device 10 and the external environment.

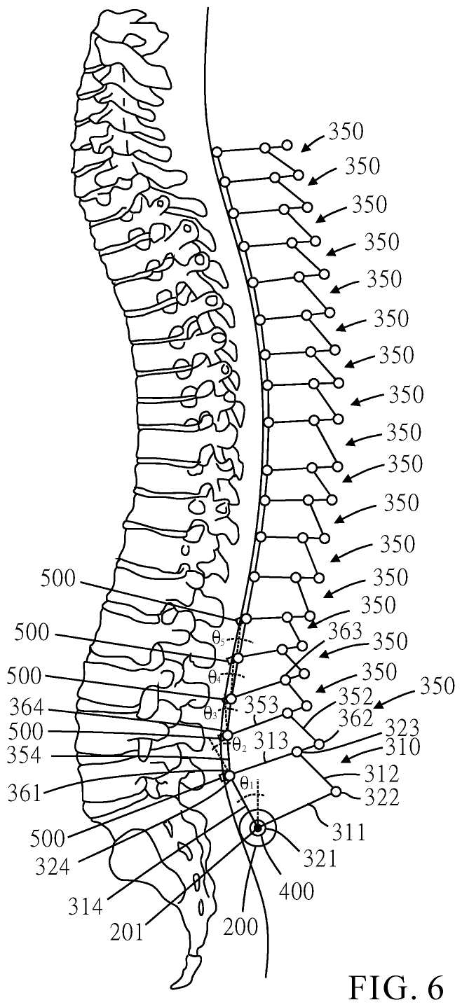

FIG. 6 is a schematic diagram illustrating the operation of an embodiment of the wearable spinal measurement device in the present disclosure. In this embodiment, a pressure sensor 500 is also disposed on the side of the fourth primary rotational axis 324 of the primary measurement unit 310 opposite to the third primary linkage 313 (i.e., the side that is close to the user's back when the wearable spinal measurement device is worn). This sensor measures the contact pressure when the fourth primary rotational axis 324 of the primary measurement unit 310 contacts the user's back due to the operation of the actuation module 200. Similarly, on the side of the fourth secondary rotational axis 364 of each secondary measurement unit 350, opposite to the third secondary linkage 353 (i.e., the side close to the user's back when the wearable spinal measurement device is worn), there is also a pressure sensor 500. These sensors measure the contact pressure when the fourth secondary rotational axis 364 of each secondary measurement unit 350 moves in conjunction with the operation of the actuation module 200 and makes contact with the user's back. By measuring the contact pressure between each fourth secondary rotational axis 364 and the user's back, it is possible to determine whether both the primary measurement unit 310 and each secondary measurement unit 350 are in contact with the user's back. If any pressure sensor 500 does not measure contact pressure, it indicates that the measurement mechanism 300 is not perfectly adhering to the user's spine. When the processing module 600 receives a contact pressure signal from any pressure sensor 500 indicating no contact pressure, it outputs a control signal to the actuation module 200, causing the transmission element 201 to rotate further to ensure that the fourth primary rotational axis 324 of the primary measurement unit 310 and each fourth secondary rotational axis 364 of the secondary measurement units 350 all make contact with the user's back, ensuring that the measurement mechanism 300 fits perfectly on each segment of the user's spine. This achieves precise measurement of the user's spinal posture and prevents any section of the secondary measurement units 350 from being suspended. Additionally, by using pressure sensors 500, it is possible to confirm whether the primary measurement unit 310 or plurality of secondary measurement units 350 exert excessive force on the user's back due to the operation of the actuation module 200. When the processing module 600 receives a contact pressure signal indicating excessive contact pressure from a pressure sensor 500, it also outputs a control signal to reduce the operating torque of the actuation module 200 to prevent discomfort and pressure on the user's back.

In some embodiments of the present disclosure, the processing module 600 further includes a processor 601. The processor 601 calculates and outputs control signals to the actuation module 200 in real-time by receiving angle change signals from the angular sensor 400, which measure changes in angle, and contact pressure signals from the pressure sensors 500, which measure contact pressure in real-time. This real-time adjustment of the actuation module's 200 output torque ensures that the measurement mechanism 300 can respond quickly to changes in the user's posture and maintain its adherence to the user's spine. Furthermore, the processor 601 can store information about the size characteristics of the measurement mechanism 300 and perform real-time calculations of the user's spinal posture results by receiving angle change signals from the angular sensor 400 and contact pressure signals from the pressure sensors 500. This information can be output to external electronic devices without the need for additional computers, servers, or electronic equipment, allowing users or other medical professionals to obtain real-time information about the user's spinal posture and further record, analyze, and plan medical treatments.

In some embodiments of the present disclosure, the measurement mechanism 300 is an underactuated mechanism, so it only requires a single actuation module 200 to be positioned at the bottom of the measurement mechanism 300 and connected to the first primary linkage 311 of the primary measurement unit 310 to actuate the entire measurement mechanism 300. This eliminates the need for additional actuating devices, motors, or other driving devices and eliminates the need to provide power-driven devices or motors for each vertebral segment. A single actuation module 200 provides the power needed for the measurement mechanism 300 to adhere to the user's back and accurately measure spinal posture, thereby reducing device weight and achieving lightweight design objectives.

In some embodiments of the present disclosure, the angular sensor 400 is a magnetic encoder that measures angle changes without the need for complex or additional mechanisms for angle measurement, achieving a lightweight design. In this embodiment, the angular sensor 400 has an angle sensing accuracy of less than or equal to 0.1 degrees, enabling the processing module 600 and processor 601 to receive precise angle change signals and achieve accurate representation of spinal posture. The angular sensor 400 is wirelessly connected to the processing module 600, using methods such as wireless communication or Bluetooth communication, to avoid excessive wiring between the angular sensor 400 and the processing module 600, preventing inconvenience, wire tangling, and difficulties in wire arrangement. In some embodiment, wired connections are used between the angular sensor 400 and the processing module 600 for simplicity of component design.

In some embodiments of the present disclosure, the wearable spinal measurement device includes an inertial sensor disposed on the actuation module 200. The inertial sensor measures the coordinates of the actuation module 200 relative to the earth and serves as a measurement reference point for the entire wearable spinal measurement device in space. It is noted that the placement of the inertial sensor is not limited to the actuation module 200 and can be positioned anywhere on the wearable spinal measurement device to provide coordinate information for the entire wearable spinal measurement device relative to the earth.

In some embodiments of the present disclosure, the pressure sensor 500 is a flexible force-sensitive resistor (FSR). The use of flexible FSRs eliminates the need for larger, heavier pressure sensing devices, achieving lightweight design. The pressure sensor 500 is wirelessly connected to the processing module 600, using methods such as wireless communication or Bluetooth communication, to minimize the wiring between each pressure sensor 500 and the processing module 600, avoiding inconvenience, wire tangling, and difficulties in wire arrangement. In some embodiment, wired connections are used between the pressure sensor 500 and the processing module 600 for simplicity of component design.

In some embodiments of the present disclosure, the measurement mechanism 300 further includes elastic elements installed in the primary measurement unit 310 and a plurality of secondary measurement units 350 to prevent the measurement mechanism 300 from collapsing due to factors such as gravity, friction, and initial conditions when the actuation module 200 has not yet been activated.

In some embodiments of the present disclosure, the size characteristics and physical properties of the primary measurement unit 310 and each secondary measurement unit 350 within the measurement mechanism 300 are defined by a mathematical computation model. The defined parameters include the dimensions, elastic coefficients, and weights of each measuring unit. Specifically, the lengths of the connectors in each measuring unit can be different, and the elastic coefficients and weights of each measuring unit can also vary. By inputting various requirements, such as different spinal tracking length needs, varying spinal segment lengths, different spinal curvatures, and diverse measurement needs, into the mathematical computation model, corresponding size characteristic parameters and physical property parameters can be obtained to achieve the effectiveness of accurately tracking different users' spinal patterns and size characteristics, addressing their specific tracking requirements.

In some embodiments of the present disclosure, the mathematical computational model includes a continuous underactuated mechanism static model, a continuous underactuated mechanism static model including gravity, and a continuous underactuated mechanism static model integrating elastic elements, or any combination thereof, to compute optimized parameters under various conditions using a genetic algorithm. By optimizing the dimensional and physical characteristic parameters, the wearable spinal measurement device of the present disclosure, even with only one actuation module 200 and corresponding measurement units for each vertebral segment, can conform each measurement unit to the user's spinal segments without subjecting the user to excessive contact pressure. This achieves device lightweighting and precise tracking of spinal posture.

In some embodiments of the present disclosure, to reduce the overall weight, size, and manufacturing cost of the wearable measuring device, underactuated mechanisms are used in the design. This allows for the control of multiple degrees of freedom with only one actuating device (e.g., actuation module 200). In this embodiment, a four-bar linkage mechanism is used, considering each connecting element (e.g., primary linkages 311-314, secondary linkages 351-354) as rigid bodies and connecting them to analyze contact forces. In the continuous underactuated mechanism static model of this embodiment, the mathematical relationship of contact forces at each point can be derived by considering the elastic torques at both ends of nodes (e.g., primary rotational axes 321-324, secondary rotational axes 361-364), the elastic coefficients at both ends of nodes, the angular relationships at both ends of nodes, and the length relationships between each connecting element. This mathematical model allows for the design of measurement mechanisms of different sizes, and reasonable contact pressures can be calculated using the mathematical model, enabling the reverse calculation of the weights of different material objects (e.g., primary linkages 311-314, secondary linkages 351-354) in the device under reasonable contact pressure.

In some embodiments of the present disclosure, the inclusion of gravity in the continuous underactuated mechanism static model allows for the calculation of the actual conditions when users wear the wearable spinal measurement device. This provides a more accurate mathematical model for calculating reasonable contact pressures, taking into account different weight conditions of different objects. It can also be used to reverse calculate the weights of different material objects (e.g., primary linkages 311-314, secondary linkages 351-354) under reasonable contact pressure.

In some embodiments of the present disclosure, by integrating contact pressure into the continuous underactuated mechanism static model with gravity, the above different models can be integrated into a complete mathematical model. By considering contact pressure as the most important parameter, the model calculates the contact pressures at each node (e.g., first primary rotational axis 321, first secondary rotational axis 361). It distributes contact pressures at each node within a certain numerical range, ensuring that users can use the wearable spinal measurement device safely and comfortably. Furthermore, by ensuring that the contact pressures are positive, each node (e.g., first primary rotational axis 321, first secondary rotational axis 361) can be correctly fitted and make contact with the user's back, accurately reflecting the user's spinal posture and pattern. Additionally, in this embodiment, in order to prevent the measurement mechanism 300 from collapsing due to factors such as gravity, friction, and initial conditions when the actuation module 200 has not yet operated, elastic elements (e.g., elastic components) are provided at both ends of any primary or secondary rotational axes, allowing for the adjustment of elastic elements with different spring constants as needed between adjacent connecting elements, achieving a retention force and maintaining stability in the overall mechanical design.

In some embodiments of the present disclosure, based on the given condition parameters, such as the weight of connecting elements, the spring constants of elastic elements, the desired range of contact pressures, the default range of angle changes under several preset postural patterns, etc., a genetic algorithm can calculate dimensional information for each connecting element (e.g., primary linkages 311-314, secondary linkages 351-354). This information can be used to design and manufacture the wearable spinal measurement device to conform to various spinal patterns.

Through the wearable spinal measurement device of embodiments of the present disclosure, which tracks the user's spinal posture by conforming to the user's back, medical professionals can establish relevant important medical indicators for the user's spinal curve, such as thoracic kyphosis (TK), lumbar lordosis (LL), sagittal vertical axis (SVA), inflection points, and other related medical indicators defined based on continuous information on spinal posture measured in three-dimensional space, for use in clinical medicine and experimental research.

The present disclosure is described by way of the multiple embodiments above. A person skilled in the art should understand that, these embodiments are merely for describing the present disclosure are not to be construed as limitations to the scope of the present disclosure. It should be noted that all equivalent changes, replacements and substitutions made to the embodiments are to be encompassed within the scope of the present disclosure. Therefore, the scope of protection of the present disclosure should be accorded with the broadest interpretation of the appended claims.

What is claimed is:

1. A wearable spinal measurement device, comprising:
a wearing component;
an actuation module, disposed on the wearing component, wherein the actuation module includes a transmission element;
a measurement mechanism, disposed on the wearing component, wherein the measurement mechanism includes:
a primary measurement unit, comprising:
a first primary linkage, connected at one end to the transmission element of the actuation module;
a second primary linkage, pivotally connected at one end to the first primary linkage via a second primary rotational axis;
a third primary linkage, pivotally connected at one end to the second primary linkage via a third primary rotational axis;
a fourth primary linkage, pivotally connected at one end to the third primary linkage via a fourth primary rotational axis, and pivotally connected at the other end to the first primary linkage via a first primary rotational axis;
at least one angular sensor, disposed on the first primary rotational axis, for measuring the angular relationship between the first primary linkage and the fourth primary linkage; and
a processing module, electrically connected to the at least one angular sensor, wherein the processing module receives an angle change signal from the at least one angular sensor and transmits a measurement signal between the wearable spinal measurement device and an external environment.

2. The wearable spinal measurement device according to claim 1, wherein the measurement mechanism further comprises a plurality of secondary measurement units, each of the plurality the secondary measurement units comprising:
a first secondary linkage;
a second secondary linkage, pivotally connected at one end to the first secondary linkage via a second secondary rotational axis;
a third secondary linkage, pivotally connected at one end to the second secondary linkage via a third secondary rotational axis;
a fourth secondary linkage, pivotally connected at one end to the third secondary linkage via a fourth secondary rotational axis, and pivotally connected at the other end to the first secondary linkage via a first secondary rotational axis,
wherein the first secondary linkage of one of the secondary measurement units among the plurality of secondary measurement units is coaxially disposed with the third primary linkage of the primary measurement unit, and the first secondary linkages of the other secondary measurement units are coaxially disposed with the third secondary linkages of the adjacent secondary measurement units,
wherein the plurality of secondary measurement units are disposed along a line connecting the first primary rotational axis to the fourth primary rotational axis of the primary measurement unit, and
wherein there are a plurality of angular sensors, and each of the plurality of angular sensors is disposed on the first secondary rotational axis of each secondary measurement unit.

3. The wearable spinal measurement device according to claim 1, wherein the actuation module is a torsion spring.

4. The wearable spinal measurement device according to claim 2, wherein the number of the plurality secondary measurement units is configured to match the number of spinal bones of the user's spine.

5. The wearable spinal measurement device according to claim 2, wherein the number of the plurality secondary measurement units is configured to match a number of spinal bone postures to be measured.

6. The wearable spinal measurement device according to claim 2, wherein the processing module is further electrically connected to the actuation module, and the processing module outputs a control signal to the actuation module; and the actuation module is a servo motor.

7. The wearable spinal measurement device according to claim 6, further comprising:
a plurality of pressure sensors, disposed on the opposite side of the fourth primary rotational axis of the primary measurement unit relative to the third primary linkage, and disposed on the opposite side of the fourth secondary rotational axis of each secondary measurement unit relative to the third secondary linkage, for measuring contact pressure,
wherein the plurality of pressure sensors is electrically connected to the processing module, and receives the contact pressure signals from the plurality of pressure sensors by the processing module, outputs the control signal to the actuation module by the processing module, and transmits the measurement signal between the wearable spinal measurement device and an external environment.

8. The wearable spinal measurement device according to claim 7, wherein the processing module further includes a processor, by continuously receiving the angle change signals from the angular sensors and the contact pressure signals from the pressure sensors, continuously adjusts the control signal output to the actuation module to adjust the torque of the transmission element, and continuously transmits the measurement signal to an external environment in real-time.

9. The wearable spinal measurement device according to claim 2, wherein the measurement mechanism is an underactuated mechanism.

10. The wearable spinal measurement device according to claim 1, wherein the angular sensor is a magnetic encoder.

11. The wearable spinal measurement device according to claim 7, wherein the angle sensing accuracy of the angular sensor is less than or equal to 0.1 degrees.

12. The wearable spinal measurement device according to claim 1, wherein the angular sensor is wirelessly connected to the processing module.

13. The wearable spinal measurement device according to claim 1, further comprising an inertial sensor, disposed on the actuation module, for measuring the coordinates of the actuation module relative to earth.

14. The wearable spinal measurement device according to claim 7, wherein the pressure sensor is a flexible force-sensitive resistor pressure sensor.

15. The wearable spinal measurement device according to claim 7, wherein the pressure sensor is wirelessly connected to the processing module.

16. The wearable spinal measurement device according to claim 2, wherein the processing module further includes a processor configured to calculate the corresponding spinal posture based on the size information of the measurement mechanism and the angle information from the angular sensor, and transmit the spinal posture to an external environment.

17. The wearable spinal measurement device according to claim 1, wherein the size characteristics and physical properties of the measurement mechanism are calculated by a genetic algorithm based on a mathematical computational model.

18. The wearable spinal measurement device according to claim 17, wherein the mathematical computational model includes a continuous underactuated mechanism static model, a continuous underactuated mechanism static model with gravity, or a continuous underactuated mechanism static model with integrated elastic elements.
